(19) United States
(12) Patent Application Publication
(10) Pub. No.: US 2022/0015622 A1
(43) Pub. Date: Jan. 20, 2022

(54) ON-EYE IMAGE PROCESSING

(71) Applicant: Tectus Corporation, Saratoga, CA (US)

(72) Inventors: Ritu Raj Singh, Santa Clara, CA (US); Steven Bailey, Oakland, CA (US); Phillip WenHsien Chang, San Jose, CA (US); Renaldi Winoto, Los Gatos, CA (US); Michael West Wiemer, San Jose, CA (US)

(21) Appl. No.: 16/929,971

(22) Filed: Jul. 15, 2020

(51) Int. Cl. A61B 3/00; A61B 3/14; G06T 7/13; G06T 7/00; G06T 5/00; G02B 27/01

(52) U.S. Cl. CPC A61B 3/0025; A61B 3/14; A61B 3/0008; G06T 7/13; G06T 2207/20024; G06T 5/003; G02B 27/0101; G02B 27/017; G06T 2207/30041; G06T 7/0012

(57) ABSTRACT

An eye-mounted device includes a contact lens that contains a femtoimager and corresponding femtoprojector. The femtoimager captures images of a user's surrounding environment. Images captured by the femtoimager are transmitted to the femtoprojector via a signal path containing digital image processing circuitry to perform one or more image processing functions on the captured images. The image processing circuitry comprises a plurality of filters connected in series and/or parallel that are configurable to implement different types of image processing, depending on the content of the images captured by the femtoimager. Compute approximation and simplification is utilized to reduce the power consumption within the lens. Components outside the contact lens determine the type of image processing, thus reducing power consumption within the contact lens. The femtoprojector then projects the resulting images to the user's retina.

### BACKGROUND

### 1. Technical Field

This disclosure relates generally to an eye-mounted device, such as an electronic contact lens.

### 2. Description of Related Art

Eye-mounted devices with projectors can be used for virtual reality (VR) applications and/or augmented reality (AR) applications. In AR applications, the images projected by the eye-mounted device augment what the user would normally see as his external environment, for example, as overlays on the external environment.

In some cases, eye-mounted devices may contain imagers used to capture image data from the external environment. Captured images may be used to generate processed image data to be displayed to the user. However, due to their size and location of being mounted on a user's eye, there may be restrictions on the area and maximum power consumption of components on the eye-mounted device, potentially limiting the types of image processing and analysis that can be performed.

### BRIEF DESCRIPTION OF THE DRAWINGS

Embodiments of the disclosure have other advantages and features which will be more readily apparent from the following detailed description and the appended claims, when taken in conjunction with the examples in the accompanying drawings, in which:

FIG. 1 is a block diagram of an eye-mounted device with a projector (femtoprojector) and an imaging device (femtoimager).

FIG. 2A shows a user wearing an electronic contact lens containing a femtoprojector and a femtoimager.

FIG. 2B shows a magnified view of the electronic contact lens mounted on the user's eye.

FIG. 2C shows a cross sectional view of the electronic contact lens mounted on the user's eye.

FIG. 2D is a posterior view of an electronics assembly for use in an electronic contact lens.

FIG. 3 is a block diagram of the signal path circuitry between the femtoprojector and femtoimager in the contact lens.

FIG. 4 illustrates a real-world image captured by a femtoimager and an image with edge detection applied to the captured image.

FIG. 5 illustrates a real-world image captured by a femtoimager and a processed image with edge detection and brightness enhancement generated from the captured image.

FIG. 6 illustrates an example of edge detection with high and low thresholds applied to an image of a human face.

FIG. 7 illustrates an example of edge detection with high and low thresholds applied to an image of a sidewalk.

FIG. 8 is a block diagram of the signal path circuitry between the femtoprojector and femtoimager in the contact lens.

FIGS. 9A and 9B illustrate tables showing some example selected configuration parameters that may be used for to perform selected image processing functions.

### DETAILED DESCRIPTION OF THE PREFERRED EMBODIMENTS

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

An eye-mounted device includes a contact lens that contains a femtoimager and corresponding femtoprojector. The femtoimager captures images of a user's surrounding environment. Images captured by the femtoimager are transmitted to the femtoprojector via a signal path contained in the contact lens. The signal path contains digital image processing circuitry to perform one or more image processing functions on the captured images. In some embodiments, the image processing circuitry is configurable to implement different types of image processing, depending on the content of the images captured by the femtoimager. Components outside the contact lens may be used to determine the type of image processing, thus reducing power consumption within the contact lens. The femtoprojector then projects the resulting images onto the user's retina.

The image processing circuitry of the signal path performs image processing functions on the captured images to enhance the user's view of the local environment. For example, edge detection may be performed on the captured images to highlight edges of objects within the user's view, which may assist the user in being able to identify objects in their local environment. Other types of image processing functions may include enhancing a brightness or contrast of objects in the user's view. These types of functions may be especially useful for users with poor vision.

The signal path includes digital circuitry that forms a pipeline to process the images from the femtoimager. In some embodiments, the signal path includes a pipeline of convolution filters, each configured to perform a selected function, such as edge detection, blurring, sharpening, etc. In some embodiments, the signal path may further contain a linear pre-filter configured to apply a gain to received image data, to adjust a brightness or contrast of the image data. The functions performed by each filter are selected by one or more configuration parameters for the filter. By using multiple filters in series and/or parallel, a variety of different image processing functions can be performed on the captured images by changing the configuration parameters.

In some embodiments, the type of image processing performed may be based on a content of what the user is looking at. For example, depending on what type of object the user is looking at, the image processing circuitry may be configured to perform edge detection using a lower or higher edge detection threshold, to allow to increase or decrease an amount of edges detected. In some embodiments, certain types of image processing functions (e.g., brightness adjustment) may be used in certain contexts (e.g., the user is in a dark environment), as determined based upon images captured by the femtoimager.

Due to area and power restrictions of components that can be implemented on a contact lens, the signal path may further comprise a transmitter and receiver to communicate with an off-lens processing component for handling more computationally-intensive processing functions. For example, the transmitter may periodically transmit one or more captured images to the off-lens processing component, which determines a context of the captured images. The receiver receives a corresponding configuration parameter from the off-lens processing component, which is used to configure operations of the image processing circuitry of the signal path. By partitioning different types of processing functions between the on-lens circuitry and off-lens processing components, the amount of area and power consumed by the on-lens circuitry may be reduced. In addition, by separating context determination from the image processing performed on the signal path, the image processing can be performed with low latency in substantially real-time.

In some embodiments, power consumption by the image processing circuitry of the signal path may be further reduced by simplifying arithmetic operations performed by the image processing circuitry, such as by implementing multiplication with left/right bit shifters, implementing subtraction as bit inversion, etc. In addition, to reduce latency, the image processing circuitry may process captured images by streaming rows of the images rather than by storing and processing entire frames of the images.

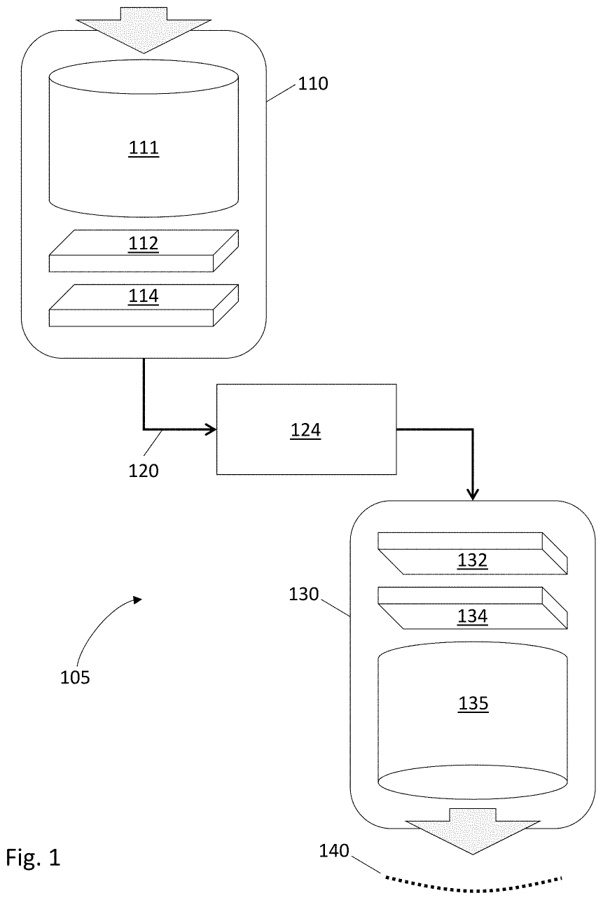

In more detail, FIG. 1 is a block diagram of an eye-mounted device 105 that includes a femtoimager 110 and a femtoprojector 130. The eye-mounted device 105 (hereinafter referred to as an electronic contact lens 105) further comprises signal paths 120 that transmit image data from the femtoimager 110 to the femtoprojector 130 through image processing circuitry 124.

The femtoimager 110 is a small imager that is outward facing and captures images of the external environment in a field of view of the femtoimager 110. The field of view of the femtoimager 110 can be the same, smaller or larger than a field of view of the user's eye. The femtoimager 110 includes imaging optics 111, a sensor array 112 and a sensor circuitry 114. The imaging optics 111 images a portion of the external environment onto the sensor array, which captures the image. The sensor array 112 may be an array of photodiodes. In some embodiments, the sensor array 112 operates in a visible wavelength band (i.e., ˜390 nm to 770 nm). Alternatively or additionally, the sensor array 112 operates in a non-visible wavelength band, such as an infrared (IR) band (i.e., ˜750 nm to 10 μm) or an ultraviolet band (i.e., <390 nm). For example, the sensor array 112 may be a thermal infrared sensor.

The sensor circuitry 114 is configured to sense and condition sensor signals produced by the sensor array 112. The sensor circuitry 114 may include analog-to-digital converters (ADC), so that the output signals are digital rather than analog. The sensor circuitry 114 may also have other functions. For example, the sensor circuitry 114 may amplify the sensor signals, convert them from current to voltage signals or filter noise from the sensor signals to keep a signal-to-noise ratio higher than a threshold value.

The imagery signals are sent along signal paths 120 from the sensor circuitry 114 through the processing circuitry 124 to driver circuitry 132 of the femtoprojector 130. The image processing circuitry 124 comprises logic circuitry to perform digital processing of the image data received from the femtoimager 110. The processing circuitry 124 may perform various types of image processing. One type of image processing is edge enhancement, where the processing circuitry 124 identifies edge boundaries in the imagery signals and increases a contrast around the identified edge boundaries. Consequently, the edge boundaries will look more defined when projected to the user. Other types of image processing may include contrast or brightness enhancement, blurring, sharpening, magnification, and the like. In some embodiments, the processing circuitry 124 may process images captured by the femtoimager 110 to generate an overlay. For example, where the femtoimager 110 operates in a thermal IR band, the processing circuitry 124 may process the image data to estimate a temperature of objects in the surrounding environment based on thermal IR image data captured by the femtoimager 110, which can be displayed to the user using the femtoprojector 130 overlaid on their existing view of the environment.

The femtoprojector 130 is a small projector that projects images corresponding to the imagery detected by the femtoimager 110 and processed by the processing circuitry 124 inward to the user's retina 140. The imagery projected by the femtoprojector 130 is visible to the user's retina 140 because the femtoprojector 130 operates at a visible wavelength band, regardless of whether the femtoimager 110 operates in a visible wavelength band, a non-visible wavelength band, or some combination thereof. The femtoprojector 130 includes driver circuitry 132, an LED (light emitting diode) array 134 and projection optics 135. In one approach, the driver circuitry 132 and LED array 134 are manufactured separately and later bonded together to form electrical connections. Alternately, they can be integrated on a single common substrate.

The driver circuitry 132 receives imagery signals from the processing circuitry 124 and converts these to drive signals to drive the LED array 134 (e.g., drive currents for LEDs). In some embodiments, the driver circuitry 132 enhances the imagery detected by the femtoimager 110, e.g., by amplifying the imagery signals. To save power, the driver circuitry 132 and LED array 134 may power down when no imagery signals are received. If the imagery signals are clocked data packets, the no signal situation may be detected when there is no clock present, for example if there is no clock signal on clock input pins or if no clock can be recovered from the incoming data stream. Also, the drive signals produced by the driver circuitry 132 may not be persistent. That is, the drive signals cause a corresponding subset of LEDs of the LED array 134 to produce light, but only when the drive signals are applied. Once the backplane no longer produces those drive signals, those LEDs also cease to produce light. In an example design, the driver circuitry 132 is an application specific integrated circuit (ASIC).

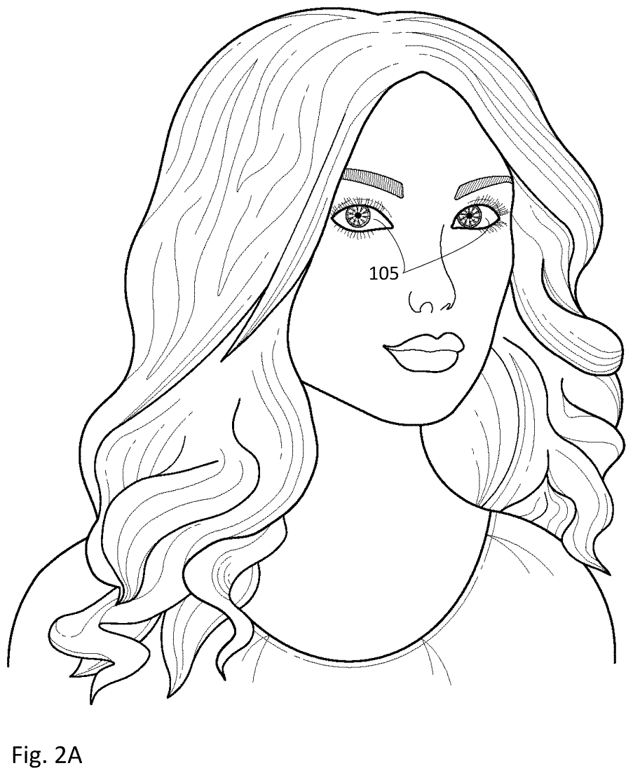

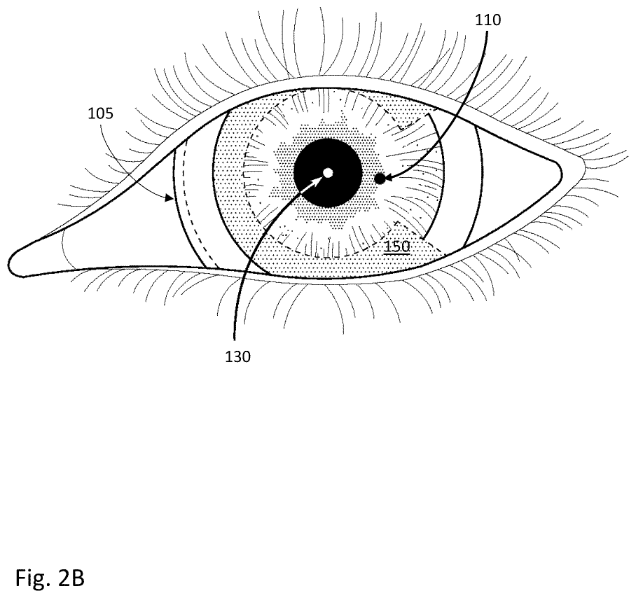

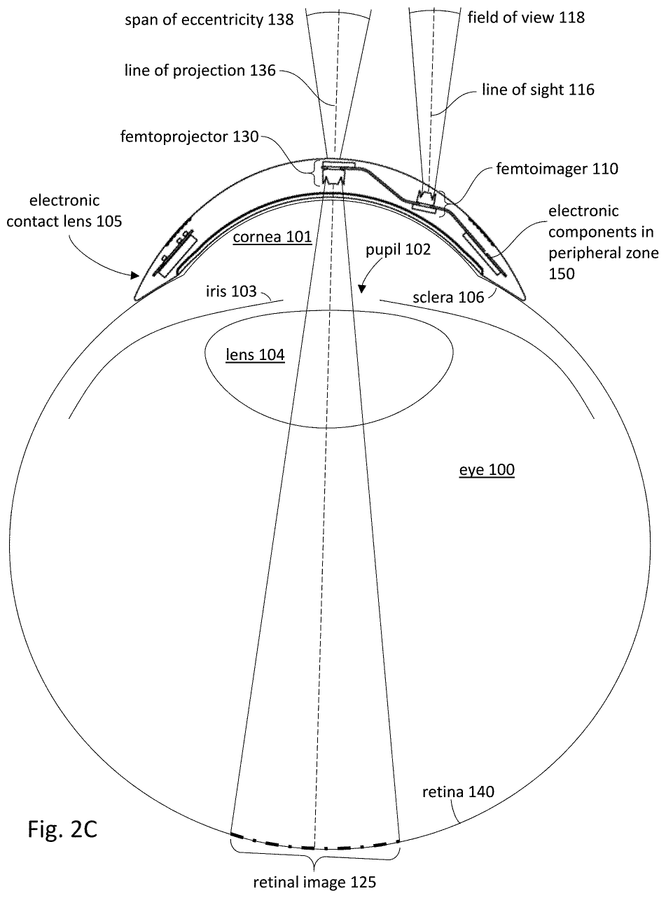

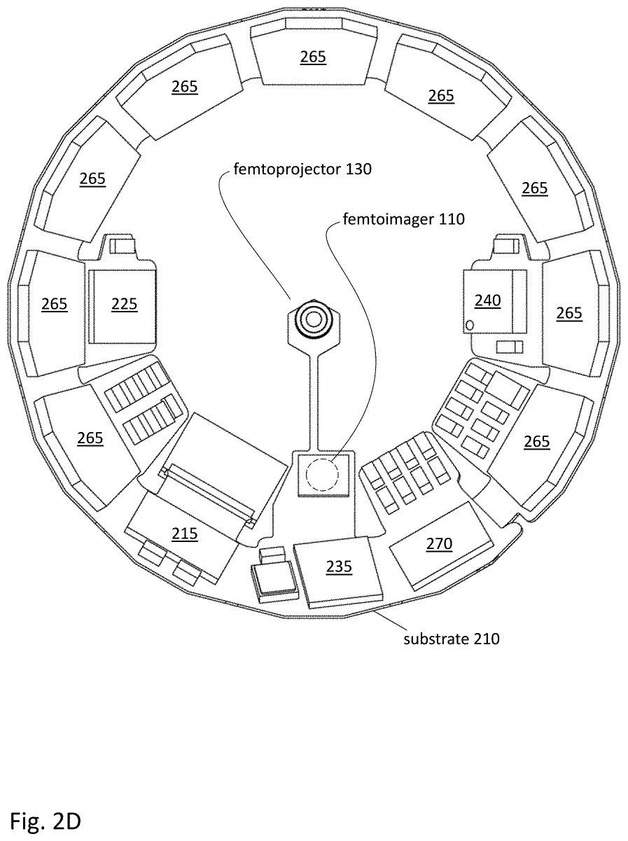

The LED array 134 contains an array of LEDs that produce light according to the drive signals from the driver circuitry 132, thus generating images corresponding to the images detected by the femtoimager 110. The array of light emitters 134 can have different geometries. One example geometry is a rectangular array of LEDs. Another example geometry is a hexagonal array of LEDs. The projection optics 135 project light from the LEDs to portions of the retina that in aggregate span a certain span of eccentricity (as described in more detail in FIG. 2C). The portion of the retina is fixed as the user's eye rotates in its socket. Thus, the femtoprojector 130 forms a visual sensation of the imagery. In some embodiments, the light from the LEDs are projected onto the retina with pixel resolutions that are highest for pixels projected to a foveal section of the retina and lower for other sections (e.g., peripheral sections) of the retina.

In some embodiments, the circuitry of the femtoimager 110, the processing circuitry 124, and the femtoprojector 130 are implemented on a single die. In other embodiments, the femtoimager 110, the processing circuitry 124, and the femtoprojector 130 are implemented on separate dies located at various locations on the electronic contact lens 105.

In some embodiments, the femtoimager 110 is regularly calibrated. In an example design, the femtoimager 110 runs a calibration cycle when the user's eyelid is closed.

The femtoimager 110 has a line of sight. The line of sight indicates a direction along which the femtoimager 110 detects imagery. The femtoprojector 130 has a line of projection, indicating a direction along which the femtoprojector 130 projects corresponding imagery to the user's retina 140. Depending on how the femtoimager 110 and the femtoprojector 130 are arranged, the line of sight of the femtoimager 110 may be parallel or not parallel to the line of projection of the femtoprojector 130. In some embodiments where the line of sight is parallel to the line of projection, the line of projection may be collinear with the line of sight. The femtoimager 110 and the femtoprojector 130 may have the same field of view/span of eccentricity and spatial resolution.

FIG. 2A shows a user wearing a pair of electronic contact lenses 105. FIG. 2B shows a magnified view of one of the electronic contact lenses 105, and FIG. 2C shows a cross sectional view of the electronic contact lens 105. The electronic contact lens 105 is worn on the surface of the user's eye. The following examples use a scleral contact lens in which the contact lens is supported by the sclera of the user's eye, but the contact lens does not have to be scleral.

As shown in FIG. 2B, the electronic contact lens 110 contains a femtoprojector 130 and a femtoimager 110. The femtoprojector 130 is located in a central region of the contact lens, so that light from the femtoprojector 130 propagates through the user's pupil to the retina, while the femtoimager 110 is located outside the central region of the contact lens so that it does not block light from entering the user's eye.

The lead line from reference numeral 105 in FIG. 2B points to the edge of the contact lens. The femtoprojector 130 and femtoimager 110 typically are not larger than 2 mm wide.

The electronic contact lens also includes other electronics, which may be located in a peripheral zone 150 of the contact lens. Electronic components in the lens may include microprocessors/controllers, motion sensors (such as accelerometers, gyroscopes and magnetometers), radio transceivers, power circuitry, antennas, batteries and elements for receiving electrical power inductively for battery charging (e.g., coils). For clarity, connections between the femtoprojector, femtoimager and electronics are not shown in FIG. 2B. Zone 150 may optionally be cut out, for example on the temporal (as opposed to nasal) side of the contact lens as shown in FIG. 2B. The electronic contact lens may include cosmetic elements, for example covering the electronics in zone 150. The cosmetic elements may be surfaces colored to resemble the iris and/or sclera of the user's eye.

FIG. 2C shows a cross sectional view of the electronic contact lens mounted on the user's eye. For completeness, FIG. 2C shows some of the structure of the eye 100, including the cornea 101, pupil 102, iris 103, lens 104, retina 140 and sclera 106. The electronic contact lens 105 preferably has a thickness that is less than two mm. The contact lens 105 maintains eye health by permitting oxygen to reach the cornea 101.

The femtoimager 110 is outward-facing, so that it “looks” away from the eye 100 and captures images of the surrounding environment. The femtoimager 110 is characterized by a line of sight 116 and a field of view (FOV) 118, as shown in FIG. 2C. The line of sight 116 indicates the direction in which the femtoimager is looking, and the field of view 118 is a measure of how much the femtoimager sees. If the femtoimager is located on the periphery of the contact lens, the contact lens surface will be sloped and light rays will be bent by refraction at this interface. Thus, the direction of the line of sight 116 in air will not be the same as the direction within the contact lens material. Similarly, the angular FOV in air (i.e., the external environment) will not be the same as the angular FOV in the contact lens material. The terms line of sight and FOV refer to these quantities as measured in the external environment (i.e., in air).

The femtoprojector 130 projects an image onto the user's retina. This is the retinal image 125 shown in FIG. 2C. This optical projection from femtoprojector 130 to retina 140 is also characterized by an optical axis, as indicated by the dashed line within the eye in FIG. 2C, and by some angular extent, as indicated by the solid lines within the eye in FIG. 2C. However, the femtoprojector typically will not be described by these quantities as measured internally within the eye. Rather, it will be described by the equivalent quantities as measured in the external environment. The retinal image 125 will appear as a virtual image in the external environment. The virtual image has a center, which defines the line of projection 136 for the femtoprojector. The virtual image will also have some spatial extent, which defines the “span of eccentricity” 138 for the femtoprojector. As with the femtoimager line of sight and FOV, the terms line of projection and span of eccentricity (SoE) for the femtoprojector refer to these quantities as measured in the external environment.

FIG. 2D is a posterior view of an electronics assembly for use in an electronic contact lens that includes some of the functionality on-lens, with some functionality implemented off-lens (i.e., outside the contact lens). The electronics assembly is approximately dome-shaped in order to fit into the contact lens. The posterior view of FIG. 2D shows a view from inside the dome. The perimeter of the dome is close to the viewer and the center of the dome is away from the viewer. The surfaces shown in FIG. 2D face towards the user's eye when the user is wearing the contact lens.

This particular design has a flexible printed circuit board 210 on which the different components are mounted. Conductive traces on the circuit board provide electrical connections between the different components. This flexible substrate 210 may be formed as a flat piece and then bent into the three-dimensional dome shape to fit into the contact lens. In the example of FIG. 2D, the components include the femtoprojector 130 and the femtoimager 110. The femtoimager 110 is facing outwards, so it is on the opposite side of the substrate 210 and is shown by hidden lines in FIG. 2D. Other components include receiver/transmitter circuitry 215, eye tracking/image stabilization circuitry 225, a display pipeline 235, attitude and heading sensors and circuitry 240 (such as accelerometers, magnetometers and gyroscopes), batteries 265 and power circuitry 270. The electronic contact lens may also include antennae and coils for wireless communication and power transfer.

The display pipeline 235 may comprise the signal paths 120 and processing circuitry 124 connecting the femtoimager 110 and femtoprojector. The display pipeline 235 may interface with the transmitter/receiver circuitry 215 to transmit/receive data to and from an external source. In some embodiments, the transmitter/receiver circuitry 215 transmits image data to the external source via an antenna (not shown). In addition, the transmitter/receiver circuitry 215 may comprise encryption circuitry configured to encrypt image data to be transmitted to the external source via the antenna. In some embodiments, the contact lens may also transmit other types data, such of eye tracking data, control data and/or data about the status of the contact lens. The display pipeline 235 receives from the external source, via the transmitter/receiver circuitry 215, control data (e.g., configuration parameters) for configuring the image processing functions of the processing circuitry 124. In other embodiments, the display pipeline 235 may receive image data from the external source.

Power may be received wirelessly via a power coil. This is coupled to circuitry 270 that conditions and distributes the incoming power (e.g., converting from AC to DC if needed). The power subsystem may also include energy storage devices, such as batteries 265 or capacitors. Alternatively, the electronic contact lens may be powered by batteries 265, and the batteries recharged wirelessly through a coil.

In addition to the on-lens components shown in FIG. 2D, the overall system may also include off-lens components outside the contact lens. For example, head tracking and eye tracking functions may be performed partly or entirely off-lens. The image processing functions of the data pipeline may also be performed partially or entirely off-lens. The power transmitter coil is off-lens, the source of image data and control data for the contact lens display is off-lens, and the receive side of the back channel is off-lens.

There are also many ways to implement the different off-lens system functions. Some portions of the system may be entirely external to the user, while other portions may be worn by the user in the form of a headpiece or glasses. Components may also be worn on a belt, armband, wrist piece, necklace, or other types of packs. In some embodiments, off-lens system functions may be performed by a plurality of off-lens components, such as an accessory device worn by the user, a remote server in communication with the accessory device, etc. For example, in some embodiments, off-lens image processing functions may be performed by a processing component on an off-lens accessory, on a remote server (e.g., a cloud server) in communication with the accessory, or some combination thereof.

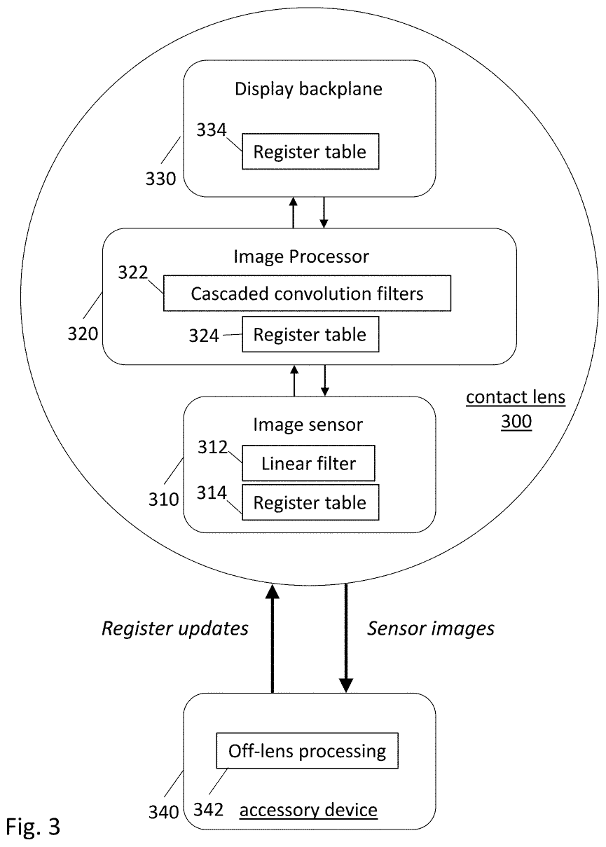

FIG. 3 is a block diagram of the signal path circuitry between the femtoprojector and femtoimager in the contact lens. The contact lens 300 comprises an image sensor 310, image processor 320, and display backplane 330. Comparing to FIG. 1, the image sensor 310 may correspond to the sensor array 112 and/or sensor circuitry 114 of the femtoimager 110, the image processor 320 may correspond to portions of the signal paths 120 and processing circuitry 124, and the display backplane 330 may correspond to the driver circuitry 132 of the femtoprojector 130.

In addition, the contact lens 300 is configured to communicate with an accessory device 340 containing an off-lens processing component 342. As discussed above, the accessory device 340 may be a device that is worn by the user, external to the user, or some combination thereof. For example, the accessory device 340 may comprise components worn by the user in the form of a headpiece, glasses, belt, armband, wrist piece, necklace, etc.

The image sensor 310 captures imagery of the user's surrounding environment, and generates image data based on the captured imagery. In some embodiments, the image sensor 310 comprises a digital linear filter 312 (also referred to as a pre-filter or a linear contrast filter) that performs one or more image processing functions on the captured image data. For example, the linear contrast filter 312 may apply a gain to the received image data, to adjust a brightness or contrast of the image data. The image sensor 310 further contains a register table 314 comprising a plurality of registers storing configuration parameters of the image sensor 310, which may comprise configuration parameters for the linear filter 312 (e.g., parameters specifying the gain applied by the linear filter 312 to the received image data, one or more thresholds used by the linear filter 312, etc.). In addition, the register table 314 may further store address mapping information (e.g., indicating addresses mapped to specific stored parameters).

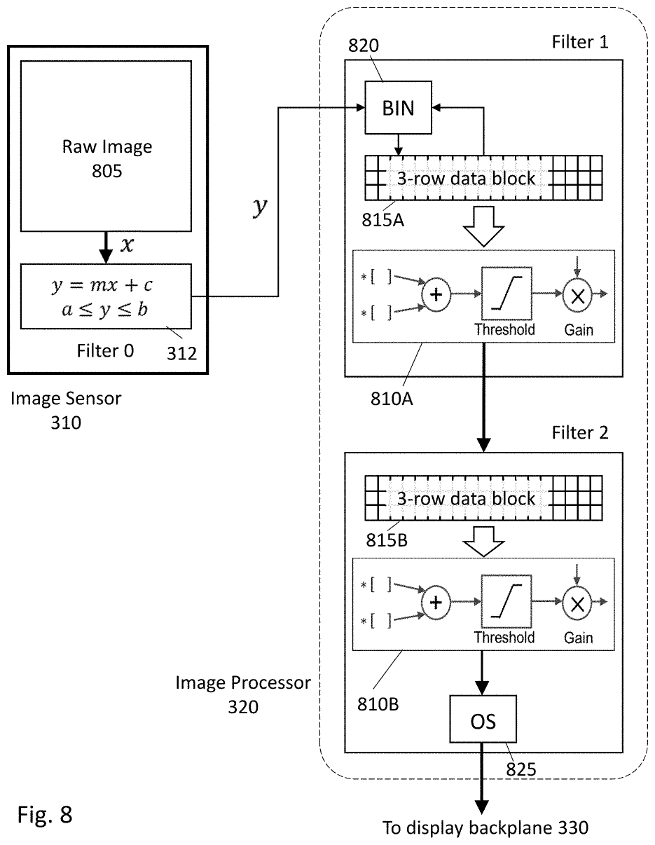

The image processor 320 processes image data received from the image sensor 310 and transmits the processed image data to the display backplane 330. The image processor 320 comprises a cascade of convolution filters 322 configured to perform image processing functions on the received image data, such as edge detection, blurring, sharpening, etc. In some embodiments, to reduce memory storage requirements and processing latency, the cascaded convolution filters 322 process the received image data by streaming rows of the images through the filters, rather than by storing and processing entire frames of the images. The cascaded convolution filters 322 comprise at least two configurable convolution filters connected in series, and are described in greater detail below in relation to FIG. 8. The image processor 310 further comprises a register table 324 that stores configuration parameters used by the cascaded convolution filters 322, such as filter kernels, threshold values, gain values, etc.

The display backplane 330 receives processed image data from the image processor 320, and generates a drive signal to drive the femtoprojector 130. The display backplane 330 may access a register table 334 that stores configuration parameters for the driver circuitry of the display backplane, such as bit mode parameters (e.g., 1 bit mode, bits per pixel parameters), gamma function parameters (non-linear input/output characteristics), etc.

While FIG. 3 illustrates each of the image sensor 310, image processor 320, and display backplane 330 as having separate register tables 314, 324, and 334, in some embodiments, the register tables may be implemented as a single memory accessible to the image sensor 310, image processor 320, and display backplane 330.

Due to area and power restrictions of components that can be implemented on a contact lens, the amount of processing that can be performed by the image processor 320 may be limited. In some embodiments, the image processor 320 comprises a transmitter and a receiver or interfaces with a transmitter/receiver (e.g., transmitter/receiver circuitry 215 in FIG. 2D) that communicates with the off-lens processing component 342 of the accessory device 340 to perform additional image processing functions in parallel with those performed by the cascaded convolution filters 322. For example, the image processor 320 may transmit image data captured by the image sensor 310 to the accessory device 340 for analysis by the off-lens processing component 342. The off-lens processing component 342 analyzes the content of the received image data to determine a type of image processing to be performed by the cascaded convolution filters 322 and/or the linear filter 312, and transmits a set of configuration parameters to the receiver of the image processor 320 for updating the register table 324 and/or register table 314. Because the off-lens processing component 342 on the accessory device 340 is not subject to the same area and power constraints of components on the contact lens 105, the off-lens processing component 342 may perform image analysis functions that are more sophisticated than those able to performed by the image processor 320 on the contact lens 310, and may store and process entire image frames at a time. In addition, the image data analysis by the off-lens processing component 342 may be performed separately and asynchronously from the image processing functions performed by the cascaded convolution filters 322 and/or the linear filter 312, minimizing additional delay between when the image data is generated by the image sensor 310 and processed image data is displayed by the femtoprojector 110. Although FIG. 3 illustrates the off-lens processing component 342 as being implemented as part of the accessary device 340, in some embodiments, the off-lens processing component 342 (or a portion thereof) may be implemented as part of a remote server or other component in communication with the accessory device 340. As such, the additional image processing functions may be performed on the accessory device 340, on a separate server, or some combination thereof.

In some embodiments, the image processor 320 transmits image data to the accessory device 340 periodically at a frame rate that is slower than a frame rate at which the images are captured by the image sensor 310. For example, the image processor 320 may be configured to transmit every Nth image captured by the image sensor, where N is an integer greater than 1. In some embodiments, the rate of image transmission is predetermined, based upon analysis of previously transmitted images, one or more user inputs, or some combination thereof. For example, in some embodiments, the off-lens processing component 342 determines a frequency parameter based upon analysis of one or more previously received images, which is received by the image processor 320 and used to control frequency of image transmission to the accessory device 340 (i.e., selection of N). In addition, in some embodiments, the image processor 320 further comprises an encryption module (not shown) configured to secure and encrypt image data to be transmitted to the accessory device 340.

In some embodiments, the image processor 320 transmits (encrypted) image data corresponding to a captured image frame to the accessory device 340 responsive to a received trigger. The trigger may correspond to an action by the user, such as a pre-determined sequence of eye blinking (e.g., three eye blinks in sequence or within a predetermined amount of time). Other types of triggers may comprise a user action performed on a component communicating with the electronic contact lens 105, change in lighting in the user's surrounding environment, recognition of an external image or pattern (e.g., a Quick Response code), and so on.

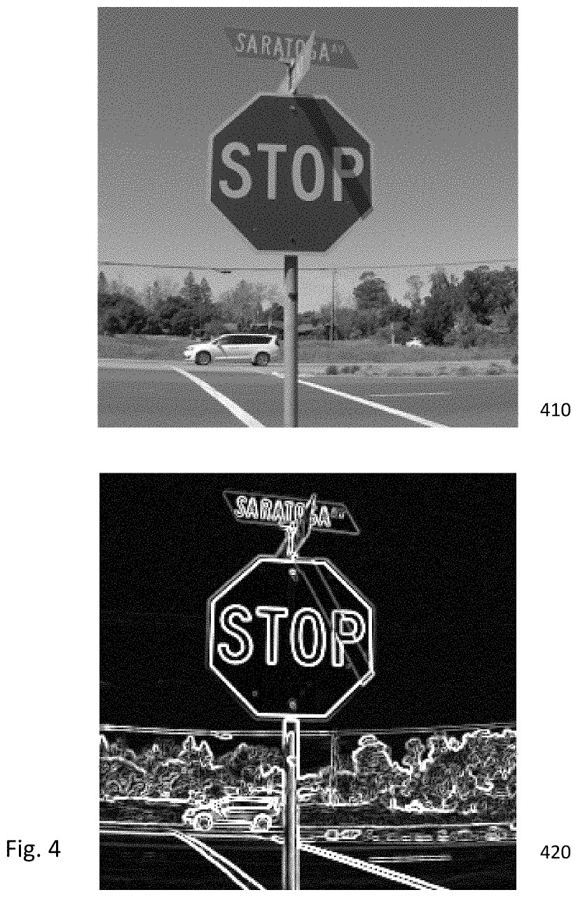

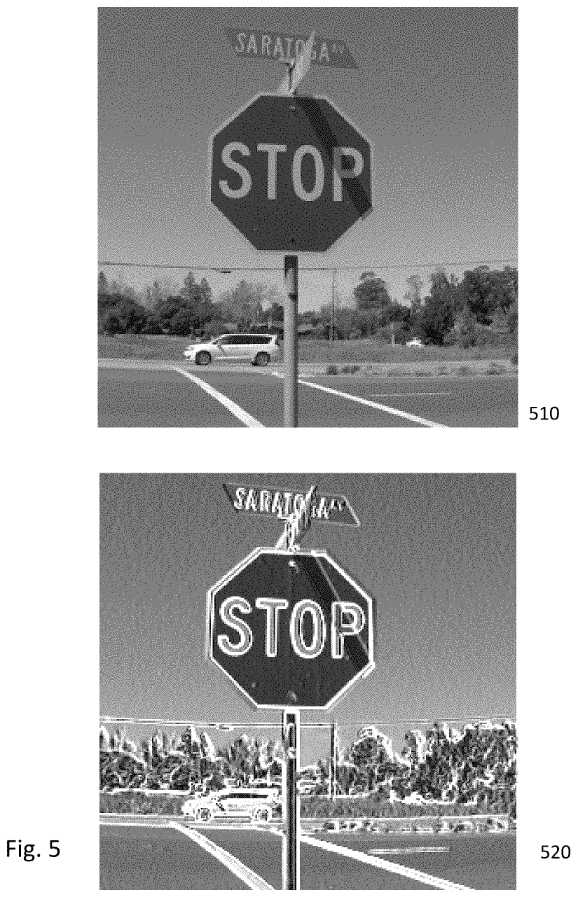

Examples of different types of image processing functions that may be performed by the linear filter 312 and the cascaded convolution filters 322 are illustrated in FIG. 4 and FIG. 5. FIG. 4 illustrates a real-world image captured by a femtoimager and edge detection applied to the captured image. Image 410 is a real-world image of a stop sign that may be captured by the femtoimager 110, in accordance with some embodiments. Image 420 illustrates a result of processing the image 410 through the cascaded convolution filters configured to perform edge detection. As illustrated in FIG. 4, edges within the processed image 420 corresponding to objects in the image 410 are highlighted. This may assist users in being able to identify objects in their local environment, especially users with low vision (e.g., vision problems that cannot be corrected through the use of corrective lenses) who may have difficulty in making out the edges of objects. In AR applications where the contact lens is at least partially transmissive, the images projected by the femtoprojector 130 may be overlaid on the user's existing vision of the surrounding environment through the contact lens. As such, when the femtoprojector 130 projects the image 420 to the eye to the user, the user may view the edge detection 420 overlaid on the image 410.

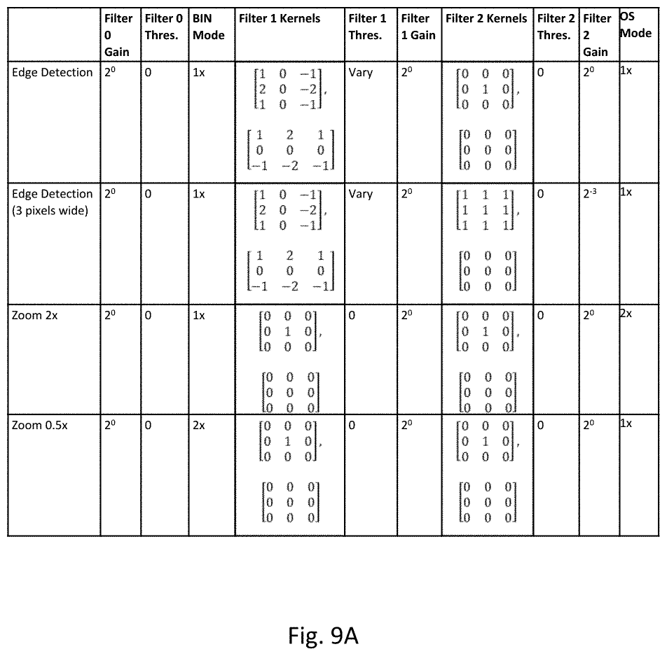

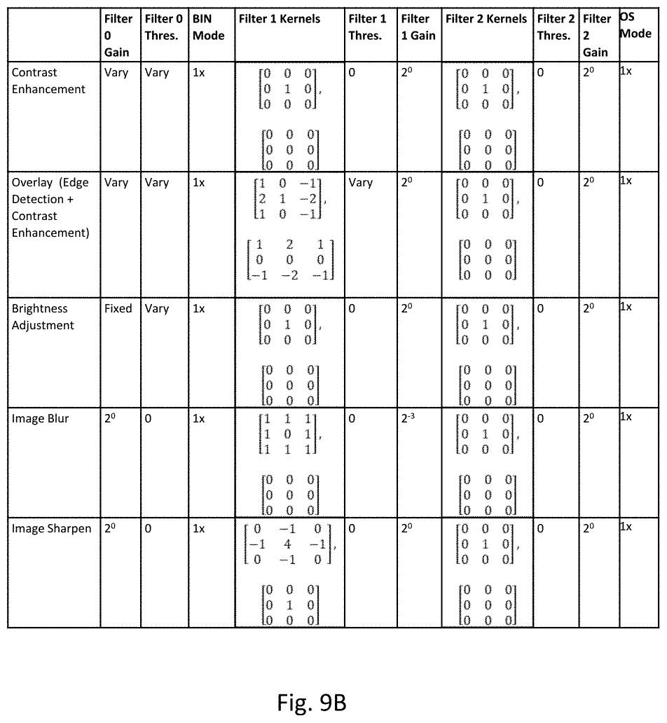

In some embodiments, the linear filter 312 and cascaded convolution filters 322 perform multiple image processing functions on captured image data. For example, each filter may be configured to perform a different function on the image, and pass the results of the processing to the next filter on the path, in accordance with a set of configuration parameters stored in register tables 314/324, as explained in greater detail in FIG. 8 and FIGS. 9A and 9B. FIG. 5 illustrates a real-world image captured by a femtoimager and a processed image with edge detection and brightness enhancement generated from the captured image. Image 510 is a real-world image of a stop sign that may be captured by the femtoimager 110, in accordance with some embodiments. Image 520 illustrates a result of processing the image 510 through a linear filter that increases a contrast level of the image and a convolution filter that performs edge detection on the image data output by the linear filter. As such, image 520 corresponds to edge detection overlaid on a contrast-enhanced version of the captured image 510. The processed image 520 may be displayed to the user overlaid on the user's real-world vision (e.g., corresponding to image 510) to increase an amount of contrast in the user's view and to highlight object edges to the user.

The image processor 320 uses the linear filter 312 and the cascaded convolution filters 322 to perform different combinations of image processing functions on captured image data, depending on the set of configuration parameters. In some embodiments, the configuration parameters are determined based on content of the captured images. For example, as discussed above in relation to FIG. 3, the off-lens processing component 340 may periodically receive images captured by the image sensor 310, and analyze the received images to determine a set of configuration parameters to be used by the linear filter 312 and the cascaded convolution filters 322. The configuration parameters may specify a type of image processing function to be performed, or alter one or more characteristics of the image processing function (e.g., threshold values, gain values, etc.).

For example, when performing edge detection, an edge detection threshold is used to identify potential edges within captured image data. Depending on a content of the received images (corresponding to what the user is looking at), the edge detection threshold may be adjusted to decrease or increase an amount of detail captured in the highlighted edges of the processed image data (e.g., a lower threshold shows more edges including weaker edges, while a higher threshold emphasizes only stronger edges).

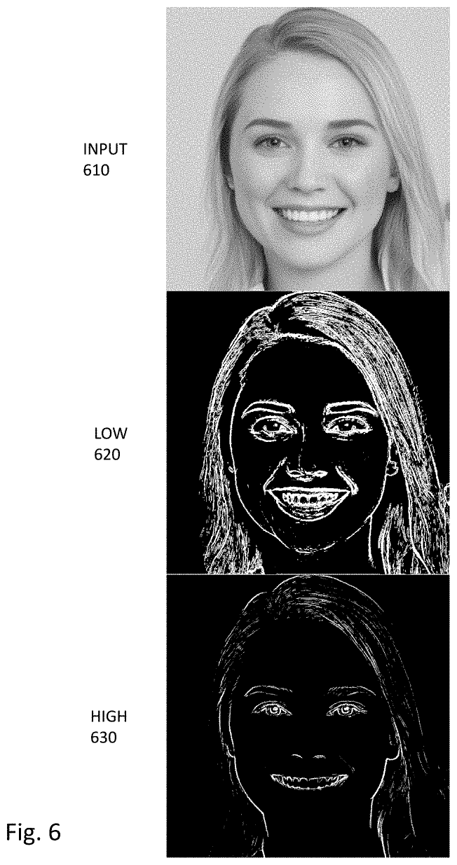

FIG. 6 illustrates an example of edge detection with high and low thresholds applied on an image of a human face. Image 610 is a real-world image of a human face that may be captured by the femtoimager 110, in accordance with some embodiments. Image 620 illustrates a result of processing the image 610 through the cascaded convolution filters that performs edge detection with a low edge detection threshold, while image 630 illustrates a result of processing the image 610 through the convolution filters using a high edge detection threshold. As shown in image 620, when the edge detection threshold is lower, a greater number of edges corresponding to the person's facial features pass the threshold, resulting in a greater level of detail in the processed image data. On the other hand, as shown in image 630, when the edge detection threshold is higher, many features of the face are not identified as edges, reducing a level of detail in the processed image data.

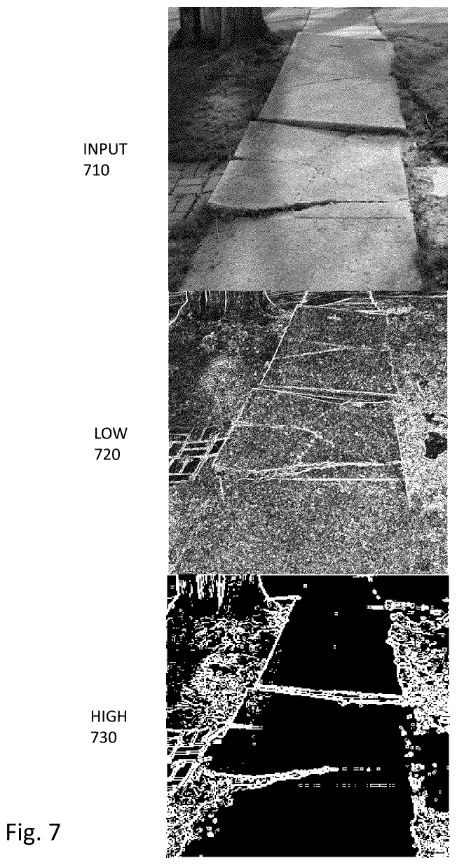

FIG. 7 illustrates an example of edge detection with high and low thresholds applied on an image of a sidewalk. Image 710 is a real-world image of a sidewalk that may be captured by the femtoimager 110, in accordance with some embodiments. Image 720 illustrates a result of processing the image 710 through a convolution filter performing edge detection with a low edge detection threshold, while image 730 illustrates a result of processing the image 710 through the convolution filter performing edge detection using a high edge detection threshold. As shown in image 720, when the edge detection threshold is low, many minor features on each sidewalk block are detected, due to the uneven texture of the pavement. On the other hand, as shown in image 730, when the edge detection threshold is high, many of the edges corresponding to these features are omitted, making it easier to discern the general shape of each sidewalk block in the resulting processed image data, while still capturing larger discontinuities (e.g., cracks, ledges) that may potentially trip up a pedestrian.

As such, different edge detection thresholds may be more useful to the user when performing edge detection on received image data, depending on what the user is looking at (e.g., a face or a sidewalk). The off-lens processing component 342 may analyze received image data to determine what type of scene the user is looking at, and set an edge detection threshold parameter accordingly. In some embodiments, the off-lens processing component 342 determines a “context” of what the user is looking at, and maps the context to a predetermined edge detection threshold value.

The off-lens processing component 342 may set different combinations of configuration parameters depending on the content of the received image data. For example, contrast enhancement may be useful for night vision applications. In some embodiments, the off-lens processing component 342 analyzes received image data to determine whether the user is in a daytime or nighttime environment (e.g., based on brightness levels of the received image data), and sets the configuration parameters for performing contrast enhancement accordingly. In some embodiments, where the image processor 320 applies a contrast and/or brightness function on the images, configuration parameters such as filter gain and threshold value may be determined based upon the content of the images, as determined by the off-lens processing component 342. In addition, in some embodiments, different types of contrast enhancement may be performed (e.g., enhancing contrast between different colors). An image may be sent to the off-lens processing component 342 to determine what form of contrast enhancement is needed in a given situation. For example, if red text on a green background is presented to a user who cannot distinguish red from green, then the red text could be highlighted in some other color or white. In some embodiments, a filter threshold may be set to a pixel value between green and red, such that pixels with red values are amplified, while green pixels are attenuated.

The off-lens processing component 342 may, by analyzing one or more images captured by the femtoimager, identify specific types of objects the user is looking at (e.g., faces), and/or determine a setting of the user (outdoor or indoor scene, daytime or nighttime, whether the user is driving a car or is just a passenger, etc.). In some embodiments, the off-lens processing component 342 may receive additional information (e.g., GPS location information, eye tracking information) from the contacts lens or from other devices to determine a context of what the user is looking at. Based on the content of the analyzed images, the off-lens processing component 342 determines a set of configuration parameters for the filters of the processing circuitry of the content lens, to be received and stored in the register tables of the content lens. The configuration parameters can be adjusted automatically as the off-lens processing component 342 analyzes additional received images.

The electronic contact lens is thus configured such that image processing functions (e.g., edge detection, contrast enhancement, etc.) are performed using the processing circuitry of the signal path on the contact lens, while image analysis functions are performed by an off-lens processing component. This partitioning of computing functions allows for computationally simpler image processing functions to be performed entirely on the lens, reducing a latency between image capture and projection. On the other hand, computationally more complex image analysis functions are performed on a separate device (e.g., accessory device 340), where minimizing power consumption and latency is less of a concern.

FIG. 8 is a block diagram of the signal path circuitry between the femtoprojector and femtoimager in the contact lens, in accordance with some embodiments. As illustrated in FIG. 8, captured raw image data is processed by a plurality of filters arranged in series. The filters comprise logical circuitry that performs digital processing of the image data along the signal path to reach the femtoprojector to be projected to the user.

The image sensor 310 captures raw image data 805 that is received by the linear filter 312 (Filter 0, or linear pre-filter). The linear filter 312 scales the pixel values of the received image data x by a gain value m (as well as a bipolar offset value c) to produce processed image data pixel values y. In some embodiments, the scaling performed by the linear filter 312 is limited by lower and upper threshold values a and b, ensuring that the processed image data pixel values y remain within certain bounds.

The output of the linear filter 312 is received by cascaded convolution filters of the image processor 320. As illustrated in FIG. 8, the cascaded convolution filters comprise at least a first convolution filter 810A (Filter 1) and a second convolution filter 810B (Filter 2) connected in series. The convolution filters 810 are configurable to perform any of a predefined set of image processing functions, including at least an edge detection function, a brightness adjustment function, an image sharpening function, and an image blur function, as determined by received configuration parameters. In some cases, only a subset of the convolution filters 810 are needed to perform a particular image processing function, in which case a filter that is not needed for the image processing function is configured to output image data in the same state as it was received, effectively bypassing the filter. Although FIG. 8 illustrates the convolution filters in a series configuration, it is understood that in some embodiments, the image processor 320 may comprise convolution filters arranged in a parallel configuration, or a series/parallel configuration.

As each convolution filter 810 processes received image data using one or more filter kernels that takes into account the neighboring pixels of each pixel of image data (e.g., for each pixel, calculating a sum of products of surrounding image data with a filter kernel), each convolution filter 810 receives image data from a respective data block 815 configured to store a plurality of rows of received image data, the number of rows corresponding to a filter kernel size (e.g., 3 rows for a 3×3 kernel). The data blocks 815 function as buffers that allow the convolution filters 810 to process the images by streaming rows of the images, rather than storing and then processing entire image frames.

Each convolution filter 810 comprises logic circuitry to convolve received image data using up to two filter kernels (e.g., a horizontal filter kernel and a vertical filter kernel) by calculating a sum of products of the image data and the filter kernels, and addition circuitry to sum the results of the two convolutions (e.g., convolution with horizontal filter kernel and convolution with vertical filter kernel). In addition, the convolution filter 810 contains threshold comparison circuitry to compare the convolved image data a threshold value to filter out values below the threshold. For example, when used for edge detection, the threshold value corresponds to the edge detection threshold, and filters out potential edges that do not meet the threshold. The convolution filter 810 may further contain gain circuitry to apply a gain to the remaining image data (e.g., to brighten detected edges).

In some embodiments, the signal path further comprises binning circuitry 820 (BIN) positioned before the convolution filters 810 configured to reduce a size of the received image data based on a binning factor. In some embodiments, the binning circuitry 820 samples every n-th pixel of the received image data along each dimension, where n corresponds to the binning factor. In some embodiments, the binning circuitry 820 determines an aggregate value of a cluster of pixels (e.g., a summation or average). For example, in an embodiment where the binning factor n is 2, the binning circuitry 820 may determine a sum or average pixel value for clusters of 2×2 pixels, effectively reducing a height and width of the output image by half along each dimension. In some embodiments, a memory (e.g., data block 815A) is used to store intermediate aggregate values produced by the binning circuitry 812 until pixel data for each cluster is received by the binning circuitry 820. The signal path may further comprise oversampling circuitry 825 (OS) positioned after the convolution filters 810, configured to increase a size of the image data based on an oversampling factor (e.g., by duplicating each pixel value m times along each dimension, where m corresponds to the oversampling factor). The binning circuitry 820 and oversampling circuitry 825 can thus be used to change the size and resolution of the image. In some embodiments, the binning circuitry 820 and oversampling circuitry 825 are used together to increase a line width of highlighted edges detected by the convolution filters 810 (e.g., by reducing the size of the image using the binning circuitry 820 prior to performing edge detection using the convolution filters 810, and then re-magnifying the image using the oversampling circuitry 825, magnifying the width of the detected edge lines). In some embodiments, the signal path may further comprise an image inversion circuit (not shown) configured to invert the pixel values of received image data.

While FIG. 8 illustrates a certain arrangement of filters in series along the signal path, it is understood that in some embodiments, different combinations of filters may be used (e.g., more than two filters along the signal path, parallel filter paths, etc.). The arrangement of filters along the signal path may be selected based upon the area and power constraints of the electronic contact lens, and to maintain a latency of less than a threshold amount.

As discussed above, the filter circuitry along the signal path is configurable to perform a variety of different image processing functions, each filter processing received image data based upon a set of configuration parameters. Configuration parameters for the linear filter 312 (Filter 0) may include a gain value (m), an offset value (c), and upper/lower threshold values (a, b). Configuration parameters for the convolution filters 810 of the cascaded convolution filters 322 (Filter 1, Filter 2) may include kernel values (e.g., horizontal kernel, vertical kernel), a threshold value, and a gain value. In addition, the binning and oversampling circuitry 820 and 825 may utilize respective configuration parameters (binning factor, oversampling factor).

FIGS. 9A and 9B illustrate tables showing some example selected configuration parameters that may be used for to perform some different image processing functions. In some embodiments, certain parameters, such as Filter 1 threshold, may be varied based upon user preferences or settings, or may be adjustable based upon one or more sensed conditions (e.g., sensed lighting conditions).

In some embodiments, the off-lens processing component 422, responsive to analyzing one or more received images, transmits to the electronic contact lens a configuration parameter indicating a type of image processing to be performed (e.g., corresponding to a row of FIG. 9A or 9B). The received configuration parameter is mapped to different image processing functions to each of the filters (e.g., Filter 0 linear filter, Filter 1 and 2 convolution filters).

By arranging the multiple filters (e.g., linear filter, at least two configurable convolution filters) in series, a variety of different image processing functions can be performed. Each filter processes image data received from the previous filter in the signal path, simplifying circuit design and reducing latency along the signal path. In cases where a particular filter is not needed to process the image data, the configuration parameters of the filter can be set to output image data in the same state as it was received (e.g., by setting gain values to 1, offset values to 0, filter kernels to identity kernels, etc.). In addition, in some embodiments, the image processor may comprise two or more filters arranged in a parallel configuration, or filters arranged in a series/parallel configuration.

In some embodiments, power consumption by the image processing circuitry of the signal path may be further reduced by simplifying arithmetic operations performed by the image processing circuitry. For example, any parameters to be used for multiplication or division (e.g., gain, filter kernel values) by the filters may be restricted to 0 or positive or negative powers of 2 (e.g., ±2°, ±2°, etc.), such that multiplication can be implemented using a bit shifter instead of a more complex multiplication circuit. Furthermore, subtraction may be implemented using bit inversion followed by addition.

By simplifying the logic circuitry for performing arithmetic operations in the linear and convolution filters, the power consumption and area of each filter may be reduced. For example, in some embodiments, each convolution filter consumes not more than 500 uW of power when processing 256 pixel by 256 pixel images at 50 FPS. In some embodiments, the linear filter/pre-filter consumes not more than 5 uW of power, or not more than 25 uW of power, when processing 256 pixel by 256 pixel images at 50 FPS.

Although the detailed description contains many specifics, these should not be construed as limiting the scope of the invention but merely as illustrating different examples. It should be appreciated that the scope of the disclosure includes other embodiments not discussed in detail above. Various other modifications, changes and variations which will be apparent to those skilled in the art may be made in the arrangement, operation and details of the method and apparatus disclosed herein without departing from the spirit and scope as defined in the appended claims. Therefore, the scope of the invention should be determined by the appended claims and their legal equivalents.

What is claimed is:

1. An eye-mounted device comprising a contact lens, the contact lens containing:
a femtoimager configured to capture images of a user's surrounding environment;
a femtoprojector configured to project images onto the user's retina;
a transmitter and receiver configured to transmit at least one of the captured images to an off-lens processing component outside the contact lens and to receive, from the off-lens processing component, configuration parameters based upon a content of the captured images; and
a signal path from the femtoimager to the femtoprojector, the signal path including image processing circuitry configured to process the images captured by the femtoimager in accordance with the configuration parameters to generate the images projected by the femtoprojector.

2. The eye-mounted device of claim 1, wherein the image processing circuitry comprises:
at least one linear filter and at least two configurable convolution filters arranged in a serial and/or parallel configuration along the signal path, wherein the linear contrast filter and convolution filters are configurable by the configuration parameters to perform any of a predefined set of image processing functions.

3. The eye-mounted device of claim 2, wherein the predefined set of image processing functions comprises an edge detection function, a brightness adjustment function, an image sharpening function, an image blur function, and a zoom function.

4. The eye-mounted device of claim 2, wherein the configuration parameters map different image processing functions to each of the least two convolution filters.

5. The eye-mounted device of claim 1, wherein the contact lens is partially transmissive, so that processed images projected by the femtoprojector are overlaid on a view of the surrounding environment transmitted through the contact lens.

6. The eye-mounted device of claim 1, wherein the transmitter and receiver is configured to transmit captured images to the off-lens processing component at a frame rate that is slower than a frame rate at which said images are captured by the femtoimager.

7. The eye-mounted device of claim 1, wherein the image processing circuitry is configured to apply an edge detection function on the images, and the configuration parameters specify an edge detection threshold value determined based upon the content of the images.

8. The eye-mounted device of claim 1, wherein the image processing circuitry is configured to apply a contrast and/or brightness function on the images, and the configuration parameters specify a filter gain and a threshold value determined based upon the content of the images.

9. The eye-mounted device of claim 1, wherein the configuration parameters are determined by the off-lens processing component.

10. An eye-mounted device comprising a contact lens, the contact lens containing:
a femtoimager configured to capture images of a user's surrounding environment;
a femtoprojector configured to project images onto the user's retina; and
a digital signal path between the femtoimager and the femtoprojector configured to digitally process the images captured by the femtoimager to generate the images projected by the femtoprojector, the signal path comprising:
at least two convolution filters arranged serially along the signal path.

11. The eye-mounted device of claim 10, wherein the convolution filters are configurable to perform any of a predefined set of image processing functions.

12. The eye-mounted device of claim 10, wherein each convolution filter convolves an input with a filter kernel, and every element of each filter kernel is either zero or a positive or negative power of two.

13. The eye-mounted device of claim 10, wherein each convolution filter calculates a sum of products of an input with a filter kernel, and each convolution filter comprises:
bit-shifting circuitry that performs the products of the input with the filter kernel.

14. The eye-mounted device of claim 10, wherein each convolution filter calculates a sum of products of an input with a filter kernel, and each convolution filter comprises:
logic circuitry that performs the entire sum of products.

15. The eye-mounted device of claim 10, wherein the signal path comprises:
logic circuitry that performs all digital processing of the images along the signal path.

16. The eye-mounted device of claim 10, wherein the signal path further comprises:
a pre-filter positioned along the signal path before the convolution filters, the pre-filter configured to apply a linear function to its input pixels.

17. The eye-mounted device of claim 16, wherein the pre-filter also limits its output pixels to maximum and minimum values.

18. The eye-mounted device of claim 10, wherein the signal path further comprises:
first circuitry positioned along the signal path before the convolution filters, the first circuitry configured to reduce a size of images input to the convolution filters; and
second circuitry positioned along the signal path after the convolution filters, the second circuitry configured to increase the size of images output by the convolution filters

19. The eye-mounted device of claim 10, wherein the convolution filters are configured to process the images by streaming rows of the images through the convolution filters rather than by storing and then processing entire frames of the images.

20. The eye-mounted device of claim 10, wherein each convolution filter consumes not more than 500 uW of power when processing 256×256 image at 50 FPS.

21. The eye-mounted device of claim 10, wherein a linear contrast filter consumes not more 5 uW of power when processing a 256×256 image at 50 FPS.
